(19) United States
(12) Patent Application Publication
(10) Pub. No.: US 2021/0254155 A1
(43) Pub. Date: Aug. 19, 2021

(54) PROBE COMPLEX FOR THE SPECIFIC DETECTION OF AT LEAST ONE SUBSTANCE, CORRESPONDING METHOD AND USE

(71) Applicant: Testo bioAnalytics GmbH, Titisee-Neustadt (DE)

(72) Inventor: Joel RIEMER, Breitnau (DE)

(73) Assignee: Testo bioAnalytics GmbH, Titisee-Neustadt (DE)

(21) Appl. No.: 17/173,482

(22) Filed: Feb. 11, 2021

(30) Foreign Application Priority Data
Feb. 14, 2020 (DE) 102020103966.2

(51) Int. Cl. C12Q 1/6876; C12Q 1/682; C12Q 1/6841; G01N 33/58

(52) U.S. Cl. CPC C12Q 1/6876; G01N 33/582; C12Q 1/6841; C12Q 1/682

(57) ABSTRACT

A probe complex (1) is provided for the specific detection of at least one substance (2), wherein the probe complex (1) binds specifically to a target segment (7) of the substance (2) and wherein an interaction with at least one detectable tag (8) can trigger a preferably optically detectable signal. The probe complex (1) includes at least one probe type in the form of a primary probe (6) having a target-specific segment (5) and at least one further probe type in the form of a secondary probe (9) having a target-specific segment (10), wherein the target-specific segments of all probe types of the probe complex (1) are matched to one another in such a way that a chain (3) of at least three probes (4) can be formed.

### INCORPORATION BY REFERENCE

German Patent Application No. 10 2020 103 966.2, filed Feb. 14, 2020, is incorporated herein by reference as if fully set forth.

### TECHNICAL FIELD

The invention relates to a probe complex for the specific detection of at least one substance, wherein the probe complex binds specifically to a target segment of the substance and wherein an interaction with at least one detectable tag can trigger a preferably optically detectable signal.

### BACKGROUND

Known methods for the specific detection of nucleic acids, i.e. DNA or RNA molecules, in individual cells include for example in-situ hybridization (ISH). This involves using short synthetic nucleic acid probes that bind to the target sequence to be detected via complementary base pairings. A variant of ISH technology in which the nucleic acid probes are labeled with fluorescent tags is fluorescence in-situ hybridization (FISH).

Molecular biology methods such as fluorescence in-situ hybridization allow the nucleic acid of a microorganism to be detected within a few hours or minutes. However, in such detection methods that are already known it is typically possible to use only target substances having a high copy number, for example rRNA, in order to obtain an adequately high signal strength. Differentiation between living and dead microorganisms is also possible, but only within the same genus or family, since different microorganisms can have different rRNA contents and can accordingly give rise to large variances in the resulting signal strengths. It is potentially also possible for rRNA to be detected from dead microorganisms. Thus, it is desirable to be able to detect sequences having a shorter half-life than rRNA, but a potentially lower copy number (for example mRNA sequences), in order to enable better living/dead differentiation. It is also desirable to detect other, potentially more specific sequences (for example species-, serotype-, division-, or pathogenicity-specific sequences) that may be present in a lower copy number (for example mRNA or DNA sequences).

In addition, there are already known enzyme-based methods that can be used to distinguish between, for example, the living/dead stage of the microorganisms to be detected. With such methods, enzyme activity is typically detectable as a “living” indicator even though the microorganisms to be detected are no longer capable of dividing. This can make reliable analysis difficult.

The advantage here is that it is possible to achieve an easy and cost-efficient analysis that is able to ensure determination of different target sequences within an organism in one process.

### SUMMARY

Against this background, it is an object of the present invention to provide a probe complex that allows specific detection of target substances, for example nucleic acid sequences or amino acid sequences, in a process that can be executed cost-efficiently and outside a laboratory setting.

The invention achieves this object through one or more of the features disclosed herein.

More particularly, in order for the invention to achieve said object with a probe complex of the type described in the introduction, it is thus proposed that the probe complex binds specifically to a target segment of the at least one substance, wherein an interaction with at least one detectable tag can trigger a preferably optically detectable signal. The probe complex of the invention is formed such that it includes at least one probe type in the form of a primary probe having a target-specific segment complementary to the target segment of the substance, and includes at least one further probe type in the form of a secondary probe having a target-specific segment complementary to the at least one primary probe, wherein the target-specific segments of the probe types are matched to one another in such a way that a chain of at least three, in particular any number of, probes can be formed.

This allows the ability to detect substances to be significantly improved, since the formation of a chain of labeled probes can allow signal amplification in optical detection. The advantage here is that the labeling process can be standardized, since it is possible for the same sequence always to be used as the secondary probe, with only the primary probe needing to be adapted to the target substance. The term “complementary” may for the purposes of the invention refer to opposite but complementary properties of an object. This could inter alia encompass also complementary binding resulting from the formation of antigen-antibody complexes and also other complementary structures of reactants that interact with one another via non-covalent bonds, as is the case for example in the DNA double helix.

Advantageous embodiments of the invention are described below, which alone or in combination with the features of other embodiments may optionally be combined together with the features noted above.

In an advantageous embodiment of the invention, the at least one probe type is a nucleic acid or an antibody or a peptide. This permits the identification and thus the detection of a substance to be achieved through the binding via complementary base pairs of the target-specific segments of the nucleic acid probes that are each formed as a chain, or through the binding of an antigen to an antibody.

In an advantageous embodiment of the invention, at least three probe types, in particular at least four probe types, are present in the probe complex. This permits the formation of a stable chain of probes that is able to ensure signal amplification in optical detection.

In an advantageous embodiment of the invention, the chain of probes is in branched or unbranched form. This permits the achievement of a flexible arrangement in the chain length and/or chain structure of the probe complex that can be adapted or optimized to the detectable substance.

In an advantageous embodiment of the invention, the probe complex includes at least one further probe type in the form of a tertiary probe having a target-specific segment complementary to the at least one secondary probe. In particular, the probe complex here is additionally formed with at least one further probe type in the form of a quaternary probe having a target-specific segment complementary to the at least one tertiary probe. Alternatively or additionally, the at least one quaternary probe is formed with a target-specific segment complementary to the at least one secondary probe. This enables a branched or unbranched chain structure to be formed between the target segment of the substance and the probe complex. This allows signal amplification and a higher intensity of fluorescence to be achieved, since binding of a plurality of secondary, tertiary, and/or quaternary probes to a primary probe can be enabled and a plurality of color tags per substance can be used.

In an advantageous embodiment of the invention, the probe types are each in the form of linear probes. Examples include mono-labeled probes, dual-labeled probes, tetra-labeled probes and multi-labeled probes. Alternatively or additionally, the probe types may each be in the form of probes having secondary structure, preferably a hairpin probe. Examples include molecular beacons and Scorpions probes. This permits a higher intensity of fluorescence and also a better signal-to-noise ratio to be achieved, which is advantageous particularly for automated use.

In an advantageous embodiment of the invention, the probe complex is labeled with at least one detectable tag. It is possible that, in the probe complex, each probe type may be labeled with the at least one detectable tag. In particular, the primary probe may be formed with at least one detectable tag. Alternatively or additionally, the probe complex may be formed with a primary probe that is free of tags plus at least one further probe type, in particular one in the form of a secondary, tertiary, and/or quaternary probe labeled with at least one detectable tag. The detectable tag may for example be an enzyme label, affinity label, and/or a dye. The dye may for example be a fluorescent dye. Optical detection is therefore achievable. The affinity label may for example include biotin-streptavidin or antigen-antibody affinity binding pairs.

In an advantageous embodiment of the invention, the target segment of the substance includes at least one DNA sequence and/or RNA sequence. In particular, the target segment of the substance may include a mRNA sequence. This permits specific detection of substances at the DNA and/or RNA level. The advantage here is that it is possible to detect within one organism not just target substances having a high copy number, for example rRNA, but also other RNAs having a low copy number, in particular mRNA sequences, tRNA sequences or DNA sequences having a low copy number, for example species-, serotype-, division-, or pathogenicity-specific sequences. The invention utilizes the ability to detect in particular housekeeping genes such as mRNA that are continuously expressed in cells capable of dividing as a consequence of being necessary for the maintenance of basic cell functions.

Alternatively or additionally, the target segment of the substance may include an amino acid sequence. This permits specific detection of substances at the peptide and/or protein level.

In an advantageous embodiment of the invention, the substance includes a nucleic acid. Alternatively or additionally, the substance may include a protein, in particular an antibody. This permits the identification and thus the detection of a substance to be achieved through the binding of an antigen to an antibody. The advantage here is that it is possible to make use of the high specificity and binding strength of the binding between antigens and antibodies. Alternatively or additionally, the substance may include a virus and/or a low-molecular-weight compound, in particular a hormone, toxin, and/or pesticide.

The invention also provides a method for performing the probe chain reaction, comprising the following steps: a) contacting a substance, preferably present in a sample, with at least one probe complex, in particular with one or more of the features described herein; b) performing a first cycle of a probe chain reaction, wherein at least one primary probe binds to at least one target segment of the substance and at least one secondary probe binds to the at least one primary probe, in particular wherein at least one tertiary probe binds to the at least one secondary probe and at least one quaternary probe binds to the at least one tertiary probe and/or to the at least one secondary probe; c) optionally performing a second cycle of a probe chain reaction, wherein at least one further probe binds to at least one of the secondary probe, tertiary probe, and/or quaternary probe; and d) preferably optically detecting the signals generated in the individual substances.

In an advantageous embodiment of the invention, the probe chain reaction(s) may for example generally be performed by a detection method based on a color reaction. In particular, the probe chain reaction(s) using the probe complex may be performed by fluorescence in-situ hybridization (FISH). Alternatively or additionally, the probe chain reaction(s) using the probe complex may be performed by immunoassay. For example, the probe chain reaction(s) may be performed by enzyme-based assay, preferably enzyme-linked immunosorbent assay (ELISA).

A preferred application provides a test kit for performing the method as claimed in one of the claims directed to a method, said test kit including at least one probe complex having one or more of the features described herein directed to a probe complex and preferably at least one reagent comprising enzyme.

A preferred application provides for a use of the probe complex according to one or more of the features described herein directed to a probe complex and/or of a method according to one or more of the features described herein directed to a method and/or of a test kit according to one or more of the features described herein directed to a test kit, for the specific detection of microorganisms and/or of their properties, in particular for the detection of at least one substance.

A preferred application provides for a use of the probe complex as claimed in a claim directed to a use, for signal amplification in optical detection by a color change, in particular by fluorescence in-situ hybridization (FISH) and/or immunoassay in which at least one probe type is present, wherein at least two probe types bind to one another in such a way that a chain of probes can be formed. For example, a use of the probe complex as claimed in a claim directed to a use may be executed for signal amplification in optical detection by enzyme-based assay, preferably enzyme-linked immunosorbent assay (ELISA). A use of the probe complex as claimed in a claim directed to a use may for example generally be executed for signal amplification in optical detection by a detection method based on a color reaction.

It is possible for the method to be performable using a fluidic channel system. For example, a fluidic channel system may take the form of a sample carrier. The sample carrier may in particular include at least one cavity containing at least one probe complex and at least one substance. Alternatively or additionally, the sample carrier may be provided with means for optically counting labeled microorganisms. The sample carrier can be designed as a disk-shaped sample carrier. For example, the sample carrier can be designed as a planar sample carrier. It is also alternatively possible to form rectangular sample carriers, as in the case of a chip card, or segment-shaped sample carriers, as in the case of a pizza slice.

The performance of the method of the invention with a fluidic channel system, preferably in the form of a sample carrier, makes it possible to achieve specific detection of at least one substance, and thus specific detection of microorganisms, in various fields of application. For example, the method of the invention can be used for microbiological analyses of foodstuffs, hygiene checks, clinical and biotechnological uses, and also for environmental analysis.

### BRIEF DESCRIPTION OF THE DRAWINGS

The invention will now be described in more detail with reference to exemplary embodiments, but without being limited to said exemplary embodiments. Further exemplary embodiments arise through combining the features of one or more claims with one another and/or with individual or multiple features of the exemplary embodiments.

In the figure(s):

FIG. 1 shows a schematic illustration of an embodiment variant of a probe complex of the invention for carrying out a method of the invention for performing the probe chain reaction.

### DETAILED DESCRIPTION

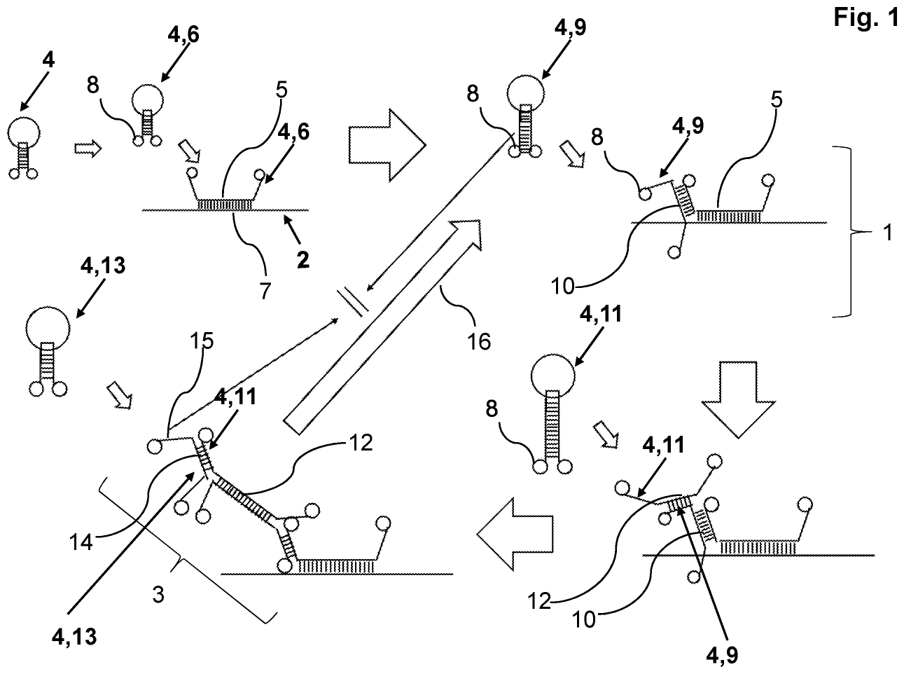

FIG. 1 shows a possible exemplary embodiment of a probe complex of the invention in simplified schematic form, where 1 refers to the probe complex of the invention in its entirety.

The probe complex 1 of the invention is designed for the execution of a specific method for the detection of at least one substance 2. The probe complex 1 here forms a chain 3 of at least three, in particular any number of, probes 4. In particular, the probe complex 1 binds via a target-specific segment 5 of a primary probe 6 to a preferably complementary target segment 7 of the substance to be detected 2.

In order for detection to be possible, the probe complex 1 is labeled with at least one detectable tag 8. Optical detection may be made possible here by for example an enzyme label, affinity label, and/or a dye.

The probe complex 1 includes at least one probe type in the form of a primary probe 6 and at least one further probe type in the form of a secondary probe 9, which respectively include target-specific segments matched to one another in such a way that a chain 3 of at least three, in particular any number of, probes 4 can be formed. The term “probe” can for the purposes of the invention refer to a nucleic acid or to an antibody or another peptide.

In order to be able to detect specific binding of the probe complex 1 to a substance 2, the substance 2 to be detected, the at least one primary probe 6, and the at least one secondary probe 9 must be linked to one another. The linking may here be established for example via complementary base pairs of the target-specific segments 5, 7, and 10 that are each produced in the form of a chain.

The at least one primary probe 6 includes the abovementioned target-specific segment 5 that binds to the target segment 7 of the substance 2. The primary probe 6 may for example be designed to allow the specific detection within one organism of RNA having a low copy number, in particular mRNA sequences, tRNA sequences, and/or DNA sequences having a low copy number, for example species-, serotype-, division-, or pathogenicity-specific sequences. Alternatively or additionally, detection of amino acid sequences, i.e. the specific detection of substances at the peptide and/or protein level, may be enabled.

FIG. 1 shows that the probes 4 in the form of nucleic acids or antibodies are each labeled with the at least one tag 8 mentioned above, through which it is possible to generate a detectable signal. The at least one secondary probe 9 has the properties that it contains a target-specific segment 10 complementary to the at least one primary probe 6. The melting temperature of the secondary probe 9 is chosen such that, under the given assay conditions, this binds to the complementary segment of the primary probe 6. This melting temperature may for example be the same as or higher than that for the hybrid of the primary probe with the target segment 7 of the substance 2.

FIG. 1 shows that the least one tertiary probe 11 may be formed such that it contains a target-specific segment 12 complementary to the at least one secondary probe 9. From FIG. 1 it can additionally be seen that the probe complex 1 may be formed with at least one further probe type in the form of a quaternary probe 13 having a target-specific segment 14 complementary to the at least one tertiary probe 11 and/or having a target-specific segment 15 complementary to the at least one secondary probe 9. This enables a branched or unbranched chain structure to be formed between the target segment 7 of the substance 2 and the probe complex 1.

The use of a chain structure between the target segment 7 of the substance 2 and the probe complex 1 enables signal amplification and a higher intensity of fluorescence, since binding of a plurality of secondary, tertiary, and/or quaternary probes to a primary probe 6 and the use of a plurality of tags 8, in particular color tags, per substance 2 can be achieved. It also enables simplified, specific detection within one organism of RNA sequences having a low copy number, in particular mRNA sequences, tRNA sequences, and/or DNA sequences having a low copy number, for example species-, serotype-, division-, or pathogenicity-specific sequences.

FIG. 1 shows an embodiment variant of a probe complex 1 comprising the probes 4, which are each in the form of hairpin probes. In a further embodiment variant that is not shown here, the probe complex 1 may be formed such that at least one probe 4 is in the form of a linear probe and because of its sequence order cannot form any internal secondary structures.

The probe complex 1 described and/or claimed herein is thus suitable in particular for use in a method for performing the probe chain reaction, in particular for the detection of at least one substance 2, by a color change, in particular by fluorescence in-situ hybridization (FISH) and/or by immunoassay as described and/or claimed herein. In particular, the probe complex 1 described and/or claimed herein is suitable for signal amplification in optical detection by fluorescence in-situ hybridization (FISH) and/or immunoassay and/or another detection method based on a color reaction.

The invention thus proposes to provide a probe complex 1 for the specific detection of at least one substance 2, wherein the probe complex 1 binds specifically to a target segment 7 of the substance 2 and wherein an interaction with at least one detectable tag 8 can trigger a preferably optically detectable signal, wherein the probe complex 1 includes at least one probe type in the form of a primary probe 6 having a target-specific segment 5 and at least one further probe type in the form of a secondary probe 9 having a target-specific segment 10, wherein the target-specific segments of all probe types of the probe complex 1 are matched to one another in such a way that a chain 3 of at least three probes 4 can be formed.

### LIST OF REFERENCE NUMBERS

1 Probe complex
2 Substance
3 Chain of probes
4 Probe
5 Target-specific segment of primary probe 6
6 Primary probe
7 Target segment of substance 2
8 Detectable tag
9 Secondary probe
10 Target-specific segment of secondary probe 9
11 Tertiary probe
12 Target-specific segment of tertiary probe 11
13 Quaternary probe
14 Target-specific segment of quaternary probe 13 complementary to the at least one tertiary probe 11
15 Target-specific segment of quaternary probe 13 complementary to the at least one secondary probe 9
16 Cycle of a probe chain reaction

1. A probe complex (1) for specific detection of at least one substance (2), the probe complex (1) being configured to bind specifically to a target segment (7) of the substance (2) and such that an interaction with at least one detectable tag (8) triggers a detectable signal, the probe complex (1) comprises at least one probe type that forms a primary probe (6) having a target-specific segment (5) complementary to the target segment (7) of the substance (2), and at least one further probe type that forms a secondary probe (9) having a target-specific segment (10) complementary to the at least one primary probe (6), wherein the target-specific segments of all probe types of the probe complex (1) are matched to one another such that a chain (3) of at least three probes (4) is formable.

2. The probe complex (1) as claimed in claim 1, wherein the at least one probe type is a nucleic acid, an antibody or a peptide.

3. The probe complex (1) as claimed in claim 1, wherein at least three of the probe types are present in the probe complex (1).

4. The probe complex (1) as claimed in claim 1, wherein the chain (3) of probes (4) is in branched or unbranched form.

5. The probe complex (1) as claimed in claim 1, further comprising at least one further probe type in the form of a tertiary probe (11) having a target-specific segment (12) complementary to the at least one secondary probe (9), and at least one further probe type in the form of a quaternary probe (13) having a target-specific segment (14) complementary to the at least one tertiary probe (11), a target-specific segment (15) complementary to the at least one secondary probe (9), or both, such that a branched or unbranched chain structure is formed between the target segment (7) of the substance (2) and the probe complex (1).

6. The probe complex (1) as claimed in claim 1, wherein the probe types are each in the form of at least one of linear probes or probes having secondary structure.

7. The probe complex (1) as claimed in claim 1, wherein the at least one detectable tag (8) is at least one of: (a) formed on at least one of the primary probe or the at least one further probe type, (b) is an enzyme label, (c) affinity label, or (d) a dye.

8. The probe complex (1) as claimed in claim 1, wherein the target segment (7) of the substance (2) includes at least one of a DNA sequence, a RNA sequence, or an amino acid sequence.

9. The probe complex (1) as claimed in claim 1, wherein the substance (2) includes at least one of a nucleic acid or a protein.

10. A method for performing a probe chain reaction, comprising the following steps:
a) contacting a substance (2) with the at least one probe complex (1) according to claim 1;
b) performing a first cycle (16) of a probe chain reaction, wherein the at least one primary probe (6) of the probe complex binds to at least one target segment (7) of the substance (2) and the at least one secondary probe (9) of the probe complex binds to the at least one primary probe (6);
c) optionally performing a second cycle (16) of a probe chain reaction, wherein at least one further probe (4) binds to the at least one secondary probe; and
d) detecting signals generated in the individual substances.

11. The method as claimed in claim 10, wherein at least one of the probe chain reactions is performed using the probe complex (1) by a color change.

12. A test kit for performing the method as claimed in claim 10, comprising the at least one probe complex and at least one reagent comprising enzyme.

13. The method of claim 10, wherein the probe complex is for specific detection of at least one of microorganisms or of properties of the microorganisms.

14. The method of claim 13, wherein the probe complex is configured for signal amplification in optical detection by fluorescence in-situ hybridization (FISH) and/or immunoassay in which at least two of the probe types bind to one another forming the chain (3) of the probes (4).

15. The probe complex (1) as claimed in claim 1, wherein the detectable signal is an optically detectable signal.

16. The probe complex (1) as claimed in claim 7, wherein the dye is a fluorescent dye or the affinity label includes biotin-streptavidin or antigen-antibody affinity binding pairs, or the dye is a fluorescent dye and the affinity label includes biotin-streptavidin or antigen-antibody affinity binding pairs.

17. The probe complex (1) as claimed in claim 9, wherein the substance (2) includes at least one of an antibody, a virus, a low-molecular-weight compound, a hormone, a toxin, or a pesticide.

18. The method of claim 10, further comprising:
in step b), at least one tertiary probe (11) binds to the at least one secondary probe (9) and at least one quaternary probe (13) binds to at least one of the at least one tertiary probe (11) or the at least one secondary probe (9), and in step c) the at least one further probe (4) binds to at least one of the secondary probe, the tertiary probe, or the quaternary probe.
